(19) United States
(12) Patent Application Publication
(10) Pub. No.: US 2021/0060560 A1
(43) Pub. Date: Mar. 4, 2021

(54) MICROFACS FOR DETECTION AND ISOLATION OF TARGET CELLS

(71) Applicant: INDIAN INSTITUTE OF TECHNOLOGY MADRAS (ITT MADRAS), Chennai (IN)

(72) Inventors: ASHIS KUMAR SEN, Chennai (IN); ABHISHEK SRIVASTAVA, Chennai (IN); RAVINDRA GAIKWARD, Chennai (IN); KARTHICK S, Chennai (IN); JAYAPRAKASH KS, Chennai (IN); ABHISHEK RAJ D, Chennai (IN); SNEHA MARIA M, Chennai (IN); PRIYANKAR SHIVHARE, Chennai (IN)

(73) Assignee: INDIAN INSTITUTE OF TECHNOLOGY MADRAS (ITT MADRAS), Chennai (IN)

(21) Appl. No.: 16/603,069

(22) PCT Filed: Apr. 5, 2018
(86) PCT No.: PCT/IN2018/050194

§ 371 Date: Oct. 4, 2019

(30) Foreign Application Priority Data
Apr. 5, 2017 (IN) 201741012180

(51) Int. Cl. B01L 3/00; G01N 15/14; G01N 33/50

(52) U.S. Cl. CPC B01L 3/502761; B01L 3/502784; G01N 15/1459; G01N 2015/0053; G01N 33/5044; G01N 2015/1413; G01N 15/1404

(57) ABSTRACT

The present invention relates to the detection and isolation of target cells based on microfluidics and cell sorting technology (MicroFACS). In this method the biological cells and microparticles are encapsulated inside hydrodynamically generated droplets and analyzed using suitable optics based on fluorescence and scattering signals. Once the target cells are detected, the optics triggers electro-coalescence for sorting of the target cells into an aqueous stream.

### FIELD OF THE INVENTION

The present invention relates to a cell sorting systems used in medical diagnoses and biological studies by employing the advancements in the field of microfluidic technology. Most specifically relates to rapid extraction of the target cells from droplets without any damage to the cells.

### BACKGROUND OF THE INVENTION

Fluorescence Activated Cell Sorter (FACS) is an instrument, which interrogates a small volume of fluid to detect and sort biological cells present in a sample fluid [J. S. Kim, et al., PAN Stanford Publishing, Singapore, 2010]. Presently, due to its capability for detailed analysis, FACS is the state of the art for biological sample analysis [R. B. L. Gwatkin., et al., Practical flow cytometry, 1994; Mol. Reprod. Dev., 1995]. FACS finds numerous applications including biomedical research for immunology, single cell analysis and molecular biology. However, conventional FACS systems are very expensive and thus are available only in centralized research facilities and major health care centres [R. B. L. Gwatkin., et al., Practical flow cytometry, 1994; Mol. Reprod. Dev., 1995]. Similarly, due to its complexity, regular maintenance and skilled expertise are required to operate the machine, analyse data and make reports. In addition, skilled technicians are required for fixing any functional failure and troubleshooting. These factors add a considerable cost to the maintenance of the machine and increase the cost per test in diagnosis using conventional FACS. In the last few years, research work has been carried out to design cost-effective, portable MicroFACS by employing the advancements in the field of microfluidic technology. However, one of the main hindrances in the development of a MicroFACS is the complicated techniques required for three dimensional focusing of biological cells flowing inside the microchannel and controlling interdistance between them in the optical window [P. K. Shivhare, et al., Microfluid. Nanofluidics, 2016]. Another challenge in the development of MicroFACS is the isolation of target cells downstream after detection. In literature, various techniques have been reported to achieve the isolation of target cells such as hydrodynamic [A. Wolff et al., Lab Chip, 2003], dielectrophoresis [D. Holmes et al., Micro Total Anal. Syst, 2004], optical [M. M. Wang et al., Nat. Biotechnol 2005] and piezoelectric [A. Wolff et al., Lab Chip, 2003]. However, such techniques require high voltage or high shear thus affecting cell viability and cell property, offer low throughput, employ complicated instrumentation and thus are not amenable to the development of a microfluidic sorter [S. H. Cho et al., Biomicrofluidics, 2010]. Also, none of these techniques are suitable for the extraction and isolation of target cells in single-cell format.

Many publications showed that an electric field has been employed for coalescence of droplets for microparticle extraction and droplet sorting [K. Ahn C et al., Appl. Phys. Lett., 2006; L. M. Fidalgo et al., Angew. Chemie, 2008; L. Mazutis et al., Lab Chip, 2012; T. Szymborski et al., Appl. Phys. Lett, 2011; A. R. Thiam et al., Phys. Rev. Lett, 2009]. Coalescence of droplets in an emulsion along the direction of the flow has been explored [Keunho Ahn et al., Appl. Phys. Lett, 2006]. Coalescence of aqueous droplets with a parallel stream of aqueous phase in a direction normal to the flow direction has also been investigated [V. Chokkalingam et al., Lab Chip, 2014]. However, the later device requires very high voltage (thousands of volts) and electric field (10⁷ V/m) thus not suitable for biological applications due to cell viability issue.

Thus the present invention relates to a technique in which cells are focused into a single-file stream and subsequently encapsulated inside droplets at a channel junction. The cell encapsulating droplets self-align toward the centre of the channel due to non-inertial lift force and move into the detection window as single-file thus solving the challenges stated above. Once the droplet-encapsulating target cells are detected, electro-coalescence is used to extract these cells either in single-cell format inside droplets or into an aqueous phase for downstream analysis

### SUMMARY OF THE INVENTION

The present invention relates to a cell sorting systems by employing the advancements in the field of microfluidic technology. Most specifically relates to rapid extraction of the target cells from droplets without any damage to the cells.

The detected droplet-encapsulating target cells are electro-coalesced to extract these cells either in single-cell format inside droplets or into an aqueous phase for downstream analysis. Wherein the aqueous droplets containing the cells are in continuous contact with the interface between the continuous phase and a co-flowing aqueous phase before entering the electric field region thus require significantly lower voltage and electric field. This approach enables rapid extraction of the target cells microparticles from droplets into a co-flowing stream of aqueous phase or in single-cell format without any damage to the cells.

In one embodiment, the present invention develops a MicroFACS for the isolation of target cells in which MicroFACS has three different modules which can be used independently for various applications and together for analysis and sorting of biological cells and microparticles. The three different modules are (i) focusing and encapsulation module, (ii) optical detection module and (iii) electro-coalescence module.

In other embodiment, the present invention provides a technique in which cells are focused into a single-file stream and subsequently encapsulated inside droplets at a channel junction. The encapsulated droplets are self-aligned toward the centre of the channel due to non-inertial lift force and move into the detection window as single-file stream.

In yet other embodiment, the present invention show that the encapsulated droplets are moved towards the detection modules where the target cells are detected using fluorescence signals and scattering signals received from labeled and non labeled cells respectively. The detected droplets are move towards electro-coalescence module. The electro-coalescence is used to sort target cells. This module consists of a microchannel with two inlets, one to introduce the immiscible continuous phase (oil) containing the droplets (containing cells or microparticles) and the other to introduce the co-flowing aqueous stream, and one or more pairs of electrodes connected to an alternating current (AC) power source. The electrical pressure is required to coalesce the droplet into fluid stream. Wherein, the droplets flowing in the immiscible continuous phase (oil) come in contact with the interface due to the positioning of the aqueous stream. The required voltage is 25 V or the corresponding electric field (10⁵ V/m) is at least two orders of magnitude smaller as compared to the existing methods.

In another embodiment, the present invention provides a method for continuous or on-demand coalescence of aqueous droplets containing target cells or microparticles with an aqueous phase for extraction of cells and microparticles from the discrete droplets and further processing of such cells or microparticles downstream. Continuous coalescence of droplets containing cells or microparticles or droplets (without any cells or microparticles) can be achieved using a continuous electric field. However, on-demand electro-coalescence requires activation of the electrodes only when a target cell, microparticle or droplet is detected in the optical detection module.

In yet another embodiment, the present invention provides a MicroFACS method by integration of the optical detection and electro-coalescence modules. The target cells or microparticles are detected optically, sorting of these target cells or microparticles into the co-flowing aqueous phase stream is achieved by triggering the electrodes in the electro-coalescence module. This method used for on-demand coalescence of droplets containing the target fluid or droplets of particular size.

### BRIEF DESCRIPTION OF THE DRAWINGS

FIG. 1 depicts (a) Schematic of the focusing and encapsulation module (b) Experimental images showing focusing of cells (c) Experimental images showing encapsulation of microparticles and cells in droplets.

FIG. 2 depicts (a) Schematic of the optical detection module (b) Experimental results showing detection of cells based on FSC, SSC and fluorescence data, image of a cell encapsulating droplet passing through the optical window is also shown.

FIG. 3 shows (a) Schematic of the electro-coalescence module (b) Experimental results showing electro-coalescence of a cell-encapsulating droplet, before coalescence the cell is encapsulated inside droplet, after coalescence the cell in the aqueous stream, voltage 25 V.

FIG. 4 shows an aqueous droplet in contact with aqueous planar interface with both containing surfactant molecules.

FIG. 5 shows droplet in contact with planar interface

FIG. 6 depicts a schematic representation of the MicroFACS (a) target cells sorted to aqueous phase (b) target cells in droplets in single-cell format.

Referring to the drawings, the embodiments of the present invention are further described. The figures are not necessarily drawn to scale, and in some instances the drawings have been exaggerated or simplified for illustrative purposes only. One of ordinary skill in the art may appreciate the many possible applications and variations of the present invention based on the following examples of possible embodiments of the present invention.

### DETAILED DESCRIPTION OF THE INVENTION

In the following detailed description, a reference is made to the accompanying drawings that form a part hereof, and in which the specific embodiments that may be practiced is shown by way of illustration. The embodiments are described in sufficient detail to enable those skilled in the art to practice the embodiments and it is to be understood that the logical, mechanical and other changes may be made without departing from the scope of the embodiments. The following detailed description is therefore not to be taken in a limiting sense.

The proposed invention relates to a cell sorting systems by employing the advancements in the field of microfluidic technology. Most specifically relates to rapid extraction of the target cells from droplets without any damage to the cells. The present invention develops a MicroFACS for the isolation of target cells in which MicroFACS has three different modules which can be used independently for various applications and together for analysis and sorting of biological cells and microparticles. The three different modules are (i) focusing and encapsulation module, (ii) optical detection module and (iii) electro-coalescence module.

### Focusing and Encapsulation Module

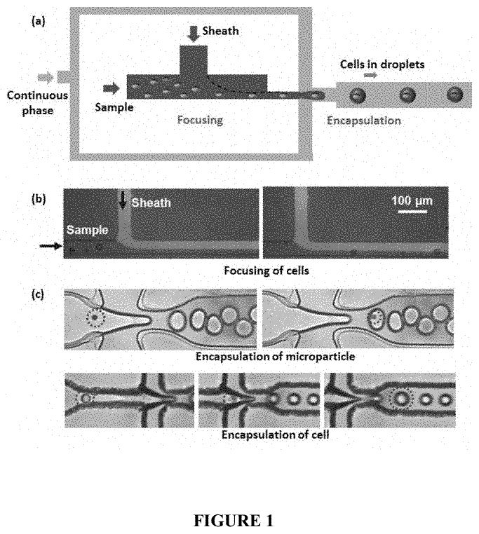

The hydrodynamic focusing and encapsulation module (FIG. 1) consists of one inlet for introducing the sample fluid (aqueous fluid containing cells or microparticles), second inlet for introducing a sheath fluid (aqueous fluid) for focusing cells or microparticles into a single-file stream, and third inlet for introducing an immiscible phase (biocompatible oil with compatible surfactant) for generating stable droplets at a flow focusing or T-junction. The hydrodynamic focusing ensures the required inter distance between any two adjacent cells or microparticles by adjusting the sheath-to-sample flow rate ratio in order to prevent clogging of the droplet generator junction and avoid encapsulation of more than one cell in a single droplet. The flow rate ratio of the discrete phase (i.e. sample+sheath) and the immiscible continuous phase (biocompatible oil) are adjusted to control the size of the droplets equal to the order of the size of the cells or microparticles. The flow rates of the sample, sheath and the continuous phase are adjusted such that the rate of arrival of cells or microparticles at the droplet junction matches with the droplet generation rate so the number of empty droplets (that do not contain cells or microparticles) is reduced.

### Optical Detection Module

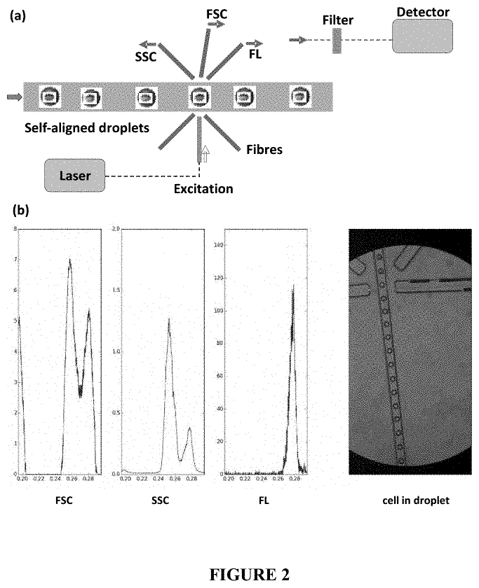

The optical detection module consists of a fluidic channel, a number of optical grooves placed at a predetermined angle with the fluid channel, laser source, fibres, filter and high-speed detectors (FIG. 2). The microchannel contains droplets encapsulating the cells and microparticles flowing in a focused self-aligned manner. The cell-encapsulating droplets migrate towards the centre of the channel due to fluidic forces (including the non-inertial lift force) and self-align. The encapsulation of the cells inside droplets and their self-alignment eliminates the need for the complicated three-dimensional focusing techniques that often limit the development of MicroFACS. To interrogate the cells or microparticles encapsulated inside droplets, laser (or other suitable light source) is used for the excitation. Fibre couples light between the laser source and the detection region in the device. The spot size of the laser beam is controlled by using suitable fibres of different size for the required collimation. When the droplets encapsulating cells (or microparticles) cross the laser beam, the optical signals are generated which are collected by the receiving fibres and captured using high speed detectors (Single Photon Counting Module-SPCM, Photomultiplier tube-PMT). If the cells or microparticles are labelled or tagged with suitable fluorophores, the fluorescence signal is captured by the detectors as the optical signature of the encapsulated cells or microparticles. Depending on the cells and the fluorophore, suitable optical filter is coupled with the collection optics to maximize the fluoresce signal. Based on the fluorescence signal, the different cells or microparticles are detected. If the cells are not labelled or tagged with fluorophores, the scattering signals are received. The detector receives the forward scatter signals of the encapsulating droplet as well as the encapsulated cells or microparticles. The forward scatter signal of the droplet is subtracted from the total scatter signal to obtain only the scatter signal of the encapsulated cells or microparticles. The forward scatter signal provides information regarding the size of the encapsulated cells or microparticles. The side scatter signal which represents the internal structure of cells or microparticles is collected and is used to distinguish between cells or microparticles for detection. By using a combination of the fluorescence, forward scatter and side scatter signatures, the target cells or microparticles are detected.

The detection module can be used for the detection of target droplets (without any cell or microparticle) that containing a fluid of interest based on the fluorescence signature of the fluid contained inside the droplet.

### Electro-Coalescence Module

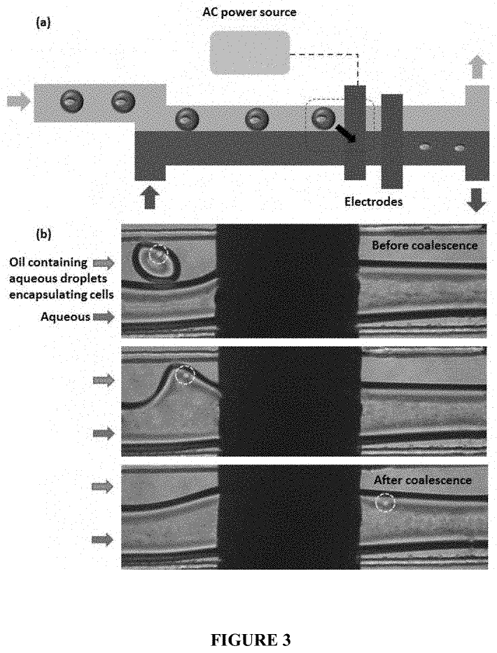

The electro-coalescence module consists of a microchannel with two inlets: one to introduce the immiscible continuous phase (oil) containing the droplets (containing cells or microparticles) and the other to introduce the co-flowing aqueous stream, and one or more pairs of electrodes connected to an alternating current (AC) power source (FIG. 3).

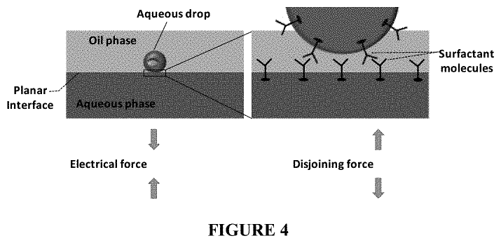

The ratio of the flow rate of the co-flowing aqueous stream is adjusted so that the droplets flowing in the immiscible continuous phase (oil) come in contact with the interface. If there is a variation in the size of the droplets, the interface location is adjusted such that even the smallest droplet comes in contact and automatically the larger droplets are in contact with the interface. In this case, an aqueous droplet and a stream of aqueous phase are separated by a very thin film of surfactant for droplet stabilization (FIG. 4) and the system is subjected to an electric field. In reported literature, a droplet and a fluid stream of the same phase (aqueous) are separated by a second phase (oil without surfactant)and when the system is subjected to an electric field, the resulting Maxwell stresses tend deform the droplet and fluid stream interface against the competing interfacial tension. As soon as the deformed droplet and fluid stream interface come in contact with each other, coalescence occurs. However, in this case since the droplet is stabilized by surfactants (in the oil phase), the electrical pressure is needed to overcome the disjoining pressure created due to the presence of surfactants for coalescence to take place, there is no deformation of droplet or interface required. The electrical pressure required to coalesce the droplet into fluid stream, when the droplet and a fluid stream interface are in contact with each other, is much smaller than the case when the stabilized droplet and a fluid stream interface are at some distance. This is because; in later case, the electrical pressure first needs to deform the droplet and the fluid stream interface to make the droplet and fluid stream contact each other and then subsequently overcome the disjoining pressure due to surfactant as well. In our case, since the droplets are already in contact with the interface due to the positioning of the aqueous stream, the required voltage (25 V) or the corresponding electric field (10⁵ V/m) is at least two orders of magnitude smaller as compared to the existing methods (thousands of volts, 10⁷ V/m) [V. Chokkalingam, Y. et al., Lab Chip, 2014].

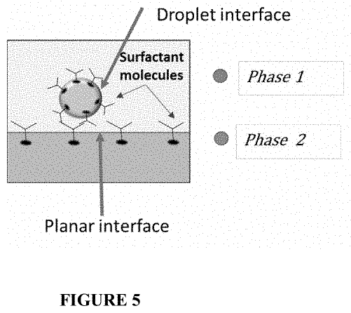

When droplet and planar-interface are stabilized by the surfactant are in contact with each other as shown in FIG. 5, it will not coalesce because surfactant molecules in two droplets will repel each other. To coalesce droplets, first have to overcome the repulsive disjoining pressure created by surfactant molecules.

To achieve coalescence the electric field has to deform the droplet and planar-interface and make the contact between interfaces. Once the contact is established the electric field has to overcome the repulsive disjoining pressure created by surfactant molecules. The electric field strength required to deform the droplets are very high compared to the electric field strength require to overcome the disjoining pressure. So the electric field required to coalesce the droplet not in contact with the other interface (˜10⁷ V/m) is one to two orders of magnitude greater compare to droplet in contact with the other interface (˜10⁵ V/m) [Liu, Z, et al., Lab on a Chip, 2014] [V. Chokkalingam Y, et al., Lab Chip, 2014]. If the droplet is in contact with the other droplet or planar interface it can be coalesced easily by applying less electric field (˜10⁵ V/m). The cell damage problems are averted completely at electric field strength less than 5×10⁵ V/m [Gascoyne P. R. C, et al., Cancers, 2014].

The method proposed here can be used for continuous or on-demand coalescence of aqueous droplets containing target cells or microparticles with an aqueous phase for extraction of cells and microparticles from the discrete droplets and further processing of such cells or microparticles downstream. The method can be used for continuous or on-demand coalescence of droplets (without any cells or particles) present in the immiscible continuous oil phase with an aqueous phase for demulsification or sorting of droplets which has importance in various applications. Continuous coalescence of droplets containing cells or microparticles or droplets (without any cells or microparticles) can be achieved using a continuous electric field. However, on-demand electro-coalescence requires activation of the electrodes only when a target cell, microparticle or droplet is detected in the optical detection module.

### Integration of the Optical Detection and Electro-Coalescence Modules

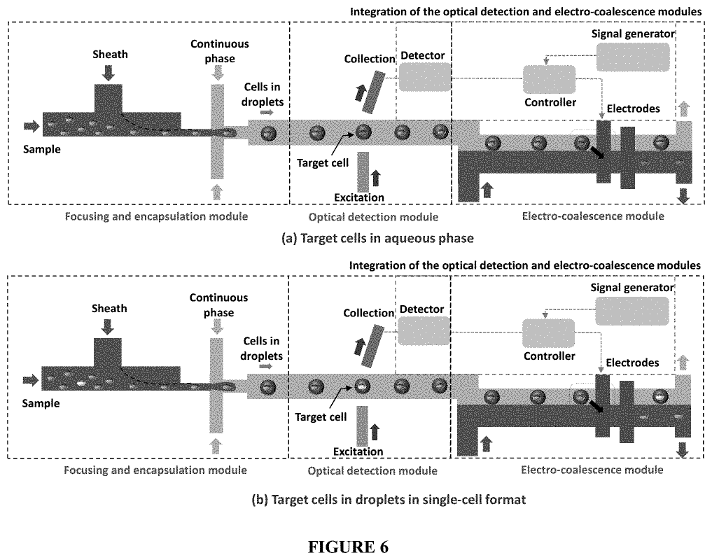

The optical detection and electro-coalescence modules are integrated to provide a MicroFACS (FIG. 6). Once the target cells or microparticles are detected optically, sorting of these target cells or microparticles into the co-flowing aqueous phase stream is achieved by triggering the electrodes in the electro-coalescence module (FIG. 6a). The optical detection and electro-coalescence units are synchronized using a microcontroller to control the switching on or off of the electrodes in the electro-coalescence region. As soon as a target cell or microparticle is detected by the optical detector, the signal is fed into the microcontroller which processes the signal and triggers the electrode. Since the velocity of the droplets in the microchannel is known, the time lag between the capture of the optical signal and the triggering of the electrodes is adjusted to accurately coalesce the droplet that contains the target cells or microparticles. The method proposed here can be used for on-demand coalescence of droplets containing the target fluid or droplets of particular size. Once such droplets are detected in the optical detection module, the electrodes can be activated for the electro-coalescence of these target droplets with the co-flowing aqueous stream.

Similarly, for applications that require single-cell analysis, the target cells encapsulated inside droplets in single-cell format can be obtained at the device outlet (FIG. 6b). In this case, the cells (other than the target cells) can be coalesced continuously by continuous application of the electric field. When a target cell is detected, the detection module sends signal to the electro-coalescence module for turning off the field so the target cells are not coalesced but flow downstream encapsulated inside droplets and collected at the outlet in single-cell format.

It may be appreciated by those skilled in the art that the drawings, examples and detailed description herein are to be regarded in an illustrative rather than a restrictive manner.

We claim:

1. A microfluidic device for analysis, sorting and demulsification of biological cells and microparticles from a complex mixture, the device comprising:
a. focusing and encapsulation module
b. optical detection module
c. electro-coalescence module
wherein the rapid extraction of the target cells or microparticles from droplets into a co-flowing stream of aqueous phase or in single-cell format without any damage to the cells,
wherein the hydrodynamic focusing and encapsulation module consists of one inlet for introducing the sample fluid, second inlet for introducing a sheath fluid for focusing cells or microparticles into a single-file stream, and third inlet for introducing an immiscible phase, wherein the flow rates of the sample, sheath and the continuous phase are adjusted in the encapsulation module such that the rate of arrival of cells or microparticles at the droplet junction matches with the droplet generation rate so the number of empty droplets is reduced
wherein the optical detection module consists of a fluidic channel, a number of optical grooves placed at a predetermined angle with the fluid channel, laser source, fibres, filter and high-speed detectors, wherein the target cells or microparticles are detected by using a combination of the fluorescence, forward scatter and side scatter signatures
wherein the electro-coalescence module consists of a microchannel with two inlets in which the aqueous droplets containing the cells are in continuous contact with the interface between the continuous phase and a co-flowing aqueous phase before entering the electric field region thus require a very low voltage and electric field

2. A method for analysis, sorting and demulsification of biological cells and microparticles from a complex mixture comprises
a. detecting the droplet-encapsulating target cells
b. extracting the droplet-encapsulated target cells either in single-cell format inside droplets or into an aqueous phase for downstream analysis using electro-coalescence
wherein, the aqueous droplets containing the cells are in continuous contact with the interface between the continuous phase and a co-flowing aqueous phase before entering the electric field
wherein, the voltage required for electro-coalescence is low in the range of 20-25 V
wherein the method is an on-demand coalescence of aqueous droplets containing target cells or microparticles with an aqueous phase for extraction of cells and microparticles from the discrete droplets
wherein the electrodes are activated only when a target cell, microparticle or droplet is detected in the optical detection module

3. A method as claimed in claim 2, wherein the cells encapsulated droplets self-align toward the centre of the channel due to non-inertial lift force and move into the detection module as single-file.

4. The microfluidic device as claimed in claim 1, wherein the forward scatter signal in the optical detection module provides information regarding the size of the encapsulated cells or microparticles.

5. The microfluidic device as claimed in claim 1, wherein the side scatter signal in the optical detection module represents the internal structure of cells or microparticles is collected and used to distinguish between cells or microparticles for detection.

6. The microfluidic device as claimed in claim 1, wherein the aqueous droplet and a stream of aqueous phase are separated by a very thin film of surfactant for droplet stabilization.

7. A method as claimed in claim 2, wherein the coalescence of encapsulated droplet and an aqueous stream occur by applying very low voltage, preferably at 25 V.

8. A method as claimed in claim 2, wherein the optical detection module is integrated with electro-coalescence module.

9. A method as claimed in claim 2, wherein the target cells or microparticles are optically detected and sorted into the co-flowing aqueous phase stream by triggering the electrodes in the electro-coalescence module.

10. A method as claimed in claim 2, wherein the method is used to isolate target cells in single-cell format without any cell damages.
